(19) United States
(12) Patent Application Publication
(10) Pub. No.: US 2024/0325772 A1
(43) Pub. Date: Oct. 3, 2024

(54) Three Dimensional Spiraling Dream Catcher

(71) Applicant: Cory James Watts, Shawnee, OK (US)

(72) Inventor: Cory James Watts, Shawnee, OK (US)

(21) Appl. No.: 18/192,045

(22) Filed: Mar. 29, 2023

(51) Int. Cl. A61N 2/00; A61N 2/02

(52) U.S. Cl. CPC A61N 2/006; A61N 2/02

(57) ABSTRACT

This invention was created to mechanically function where by its unique design and physically altered form conducts energy when near humans directly aiding in better sleep by effecting their brain wave patterns and thus effecting dreaming in proportionate relative arrays.

### CROSS-REFERENCE TO RELATED APPLICATIONS

ILLUMINATING DREAMCATCHER NOVELTY APPARATUS, Pub. No.: US 2018/0187881 A1; “[0001] This invention relates generally to novelty devices and, more particularly, to a an illuminating, hanging dream catcher novelty apparatus”. The a fore referenced novelty “dream catcher” use of material and design particular and specific to illumination. Where as this applicants invention relates generally to a mechanically functioning instrument specific for conducting energy.

### PRIOR ART

This design of dream catcher was created as an adaptive, innovative, new age method of making a functional and decorative “traditional style” craft. Traditionally, dream catcher frames were made from hoops or non-symmetrical vines etc., then wrapped with leather strips, a sinew woven netting inside, then adorned with beads and feathers. The applicant created a free form design from a metal wire and refined it into a double terminating spiraling helix. Formed wire was then wrapped with a swede lace and a net woven within it. The hand crafted decorated models of Dream-catchers that the applicant has made previously were later made in to functioning devices that conduct energy. This was not only by further refinement of its specific physical shape but by also altering its physical properties by polarizing its core wire magnetically thus enabling it to conduct the energy in near proximity to it positively aiding human synopsis. While Dream-catchers historically have been known to have spiritual importance in some cultures, in today's society the Dream-catcher has become a popular element in our culture that is appreciated for not only its novelty beauty but also considered by many people, of mixed ethnic backgrounds, to be spiritual. The development and design of this apparatus is specific for actually assisting the human brain with synopsis by conducting ions accordingly to alter brain wave patterns and state of mind wave lengths for better sleeping.

The applicants invention described herein provides for decoration, the spiritual aspects of dream-catchers, and provides the ability for one to alter their sleeping patterns to better aid sleep and gain all the benefits that come with that.

### BACKGROUND OF THE INVENTION

This invention relates generally to a mechanically functioning instrument specific for conducting energy. This “dream catcher” is a decorative and functional apparatus that originally had no physical or mechanical function. This new style of dream catcher was based on the traditional hand made craft that later was made to function by altering its physical properties enabling it to conduct electrical particles similar to the effects of earths magnetic field.

### SUMMARY OF THE INVENTION

The three dimensional spiraling dream-catcher apparatus is polarized spiral wire that terminates at the top and the bottom and conducts energy. Said wire is wrapped with a material for aesthetics as is a net woven within the spiraling frame. Beads and feathers are also draped at the top and the bottom of the spiraling frame for aesthetics as well. The frame is the critical mechanism made to function by being charged by an electrical current while grounded at one end and/or being properly stroked with a powerful magnet while it is in straight form, before re-forming it into a double terminating spiral. This polarizes the the wire and when shaped into this final form it conducts focused channels of ions in streams of negative ions flowing out one end and positive ions flowing out the other. This physical property attributed to the apparatus can be demonstrated anytime by holding a compass near it.

It is an objective of this dream catcher design to conduct energy by means of a metal wire that has been magnetically and or electrically charged and in doing so increase synapses within the human brain. Said brain stimulation has been proven to improve many mental and physical body functions safely. Additionally, research has proven that a very small magnetic field shift occurring unconsciously within the appropriate distance from the human subjects brain at the correct range of magnitude, such as the strength of earths magnetic field, there is an alteration in synapse while the subjects were in the brain state of Alpha wave length patterns causing a shift to slower wavelengths aiding sleep as well as the secretion of melatonin. Melatonin is a hormone made in the human body that regulates night and day cycles or sleep-wake cycles.

### BRIEF DESCRIPTION OF DRAWINGS

FIG. 1 is a front elevation of a wire formed into a double terminating spiraling helix that has been magnetically polarized before it is decorated in accordance with the current invention.

FIG. 2 is a top view of a wire formed into a double terminating spiraling helix that has been magnetically polarized before it is decorated in accordance with the current invention.

FIG. 3 is a front elevation of a wire formed into a double terminating spiraling helix that has been magnetically polarized after it has been decorated in accordance with the current invention.

FIG. 4 is a front elevation of a wire formed into a double terminating spiraling helix that has been magnetically polarized before it has been decorated showing the magnetic field created by magnetically polarizing the wire before it is formed into its final form.

FIG. 5 is a side view of a straight metal wire before reformed into its final shape as it is stroked with a magnet to give it a magnetic field then it is formed into the double terminating spiraling helix shape and decorated.

### DETAILED DESCRIPTION OF THE INVENTION

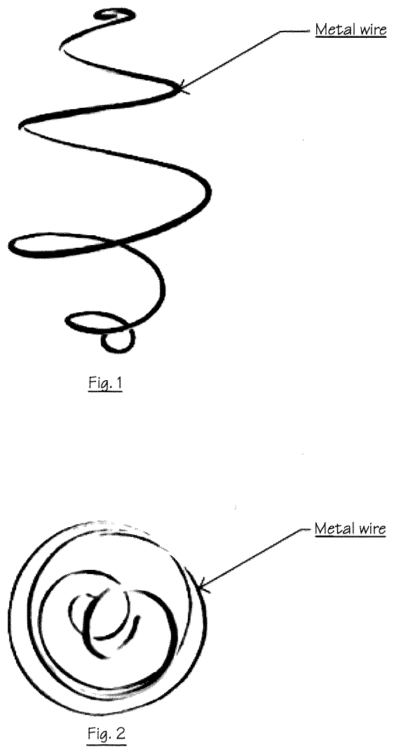

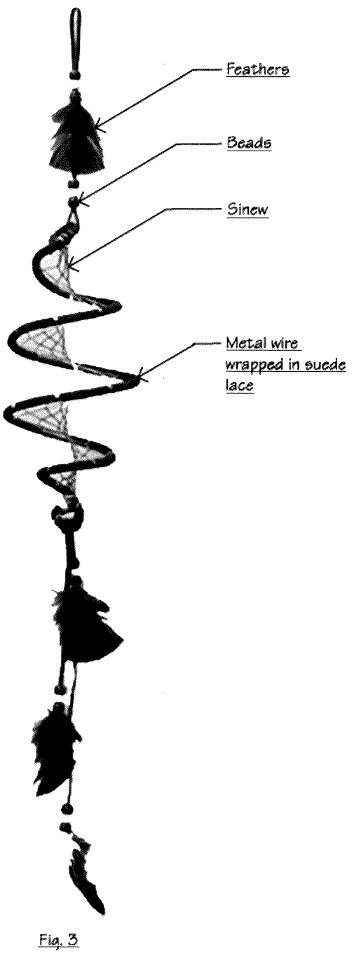

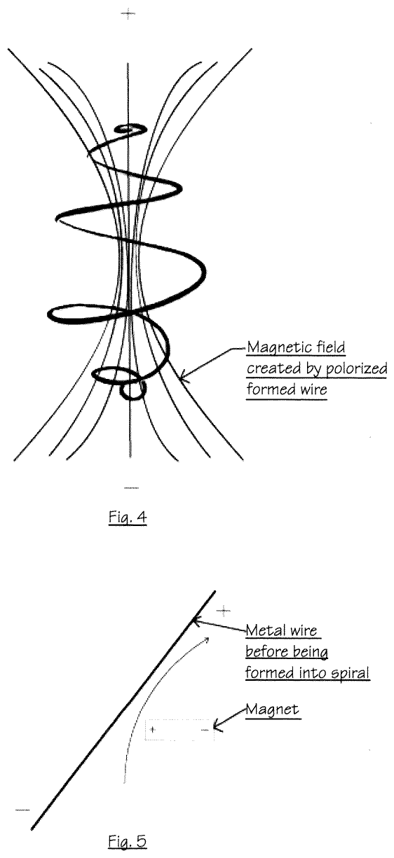

Referring now to the drawings and in particular FIGS. 1 and 2, a polarized wire shaped into a double terminating spiraling helix forms a unique and very functional energy conducting device. Now referring to FIG. 3, the energy conducting device is adorned with decorative beads, feathers, and sinew woven in the center. FIG. 4 depicts the magnetic field created by polarizing the metal wire before polarizing the wire (FIG. 5) and forming it into its spiraling design.

The design shape and physical property alteration method are specific and application there of to create this device are expressively unique and fundamental to its function and are the sole creation of the applicant.

This three dimensional spiraling dream-catcher apparatus was deigned to function by way of conducting energy near the human brain.

What is claimed:

1. An energy conducting dream-catcher apparatus, comprising: a polarized double terminating metal spiraling helix frame with netting disposed in the open center.
The energy conducting dream-catcher apparatus in claim 1, wherein said polarized double terminating metal spiraling helix frame has been given magnetic field properties enabling it to conduct energy particles. Conducting energy particles around the human brain while sleeping has been proven to help with better sleep. The publication “New evidence for a human magnetic sense that lets your brain detect the Earth's magnetic field” Published: Mar. 18, 2019 1.01 pm EDT, California Institute of Technology reported “The EEG data revealed that certain magnetic field rotations could trigger strong and reproducible brain responses. One EEG pattern known from existing research, called alpha-ERD (event-related desynchronization), typically shows up when a person suddenly detects and processes a sensory stimulus. The brains were “concerned” with the unexpected change in the magnetic field direction, and this triggered the alpha-wave reduction. That we saw such alpha-ERD patterns in response to simple magnetic rotations is powerful evidence for human magnetoreception.”
